(19) United States
(12) Patent Application Publication
(10) Pub. No.: US 2022/0409145 A1
(43) Pub. Date: Dec. 29, 2022

(54) GENERATION OF MRI IMAGES OF THE LIVER WITHOUT CONTRAST ENHANCEMENT

(71) Applicant: BAYER AKTIENGESELLSCHAFT, Leverkusen (DE)

(72) Inventors: GESINE KNOBLOCH, BERLIN (DE); CHRISTIAN LIENERTH, FRANKFURT/MAIN (DE)

(21) Appl. No.: 17/754,482

(22) PCT Filed: Oct. 5, 2020
(86) PCT No.: PCT/EP2020/077767

§ 371 Date: Apr. 4, 2022

(30) Foreign Application Priority Data
Oct. 8, 2019 (EP) 19201919.8

(51) Int. Cl. A61B 5/00; A61B 5/055; A61K 49/10

(52) U.S. Cl. CPC A61B 5/7267; A61B 5/055; A61B 5/004; A61B 5/7425; A61K 49/103

(57) ABSTRACT

The present disclosure relates to the generation of artificial MRI images of the liver. The disclosure also relates to a method, a system and a computer program product for generating MRI images of the liver without contract enhancement.

### CROSS REFERENCE TO RELATED APPLICATIONS

The present application is a U.S. national stage filing under 35 U.S.C. § 371 of International Application No. PCT/EP2020/077767, filed 5 Oct. 2020, which claims priority to European Patent Application No. EP 19201919.8, filed 8 Oct. 2019, the disclosures of each of which are incorporated in their entirety herein by this reference.

The present disclosure deals with the generation of artificial MRI images of the liver. Subjects of the present disclosure are a method, a system and a computer program product for generating MRI images of the liver without contrast enhancement.

Magnetic resonance imaging, MRI for short, is an imaging method which is used especially in medical diagnostics for depicting structure and function of the tissue and organs in the human or animal body.

In MRI, the magnetic moments of protons in an examination object are aligned in a basic magnetic field, with the result that there is a macroscopic magnetization along a longitudinal direction. This is subsequently deflected from the resting position by the incident radiation of high-frequency (HF) pulses (excitation). The return of the excited states into the resting position (relaxation) or the magnetization dynamics is subsequently detected by means of one or more HF receiver coils as relaxation signals.

For spatial encoding, rapidly switched magnetic gradient fields are superimposed on the basic magnetic field. The captured relaxation signals or the detected and spatially resolved MRI data are initially present as raw data in a spatial frequency space, and can be transformed by subsequent Fourier transformation into the real space (image space).

In the case of native MRI, the tissue contrasts are generated by the different relaxation times (T1 and T2) and the proton density.

T1 relaxation describes the transition of the longitudinal magnetization into its equilibrium state, T1 being that time that is required to reach 63.21% of the equilibrium magnetization prior to the resonance excitation. It is also called longitudinal relaxation time or spin-lattice relaxation time.

Analogously, T2 relaxation describes the transition of the transversal magnetization into its equilibrium state.

MRI contrast agents develop their action by altering the relaxation times of the structures which take up contrast agents. A distinction can be made between two groups of substances: paramagnetic and superparamagnetic substances. Both groups of substances have unpaired electrons which induce a magnetic field around the individual atoms or molecules.

Superparamagnetic contrast agents lead to a predominant shortening of T2, whereas paramagnetic contrast agents mainly lead to a shortening of T1. A shortening of the T1 time leads to an increase in the signal intensity in T1-weighted sequences, and a shortening of the T2 time leads to a decrease in the signal intensity in T2-weighted sequences.

The action of said contrast agents is indirect, since the contrast agent itself does not give off a signal, but instead only influences the signal intensity of the hydrogen protons in its surroundings.

In T1-weighted images, the paramagnetic contrast agents lead to a lighter (higher-signal) depiction of the regions containing contrast agent compared to the regions containing no contrast agent.

In T2-weighted images, superparamagnetic contrast agents lead to a darker (lower-signal) depiction of the regions containing contrast agent compared to the regions containing no contrast agent.

Both a higher-signal depiction and a lower-signal depiction lead to a contrast enhancement.

An example of a superparamagnetic contrast agent is iron oxide nanoparticles (SPIO: superparamagnetic iron oxide).

Examples of paramagnetic contrast agents are gadolinium chelates such as gadopentetate dimeglumine (trade name: Magnevist® and others), gadobenate dimeglumine (trade name: Multihance®), gadoteric acid (Dotarem®, Dotagita®, Cyclolux®), gadodiamide (Omniscan®), gadoteridol (ProHance®) and gadobutrol (Gadovist®).

Extracellular, intracellular and intravascular contrast agents can be distinguished according to their pattern of spreading in the tissue.

Contrast agents based on gadoxetic acid are characterized by specific uptake by liver cells, the hepatocytes, by enrichment in the functional tissue (parenchyma) and by enhancement of the contrasts in healthy liver tissue. The cells of cysts, metastases and most liver-cell carcinomas no longer function like normal liver cells, do not take up the contrast agent or hardly take it up, are not depicted with enhancement, and are identifiable and localizable as a result.

Examples of contrast agents based on gadoxetic acid are described in U.S. Pat. No. 6,039,931A; they are commercially available under the trade names Primovist® or Eovist® for example.

The contrast-enhancing effect of Primovist®/Eovist® is mediated by the stable gadolinium complex Gd-EOB-DTPA (gadolinium ethoxybenzyl diethylenetriamine pentaacetic acid). DTPA forms, with the paramagnetic gadolinium ion, a complex which has an extremely high thermodynamic stability. The ethoxybenzyl (EOB) radical is the mediator of the hepatobiliary uptake of the contrast agent.

Primovist® can be used for the detection and characterization of tumours in the liver. Blood supply to the healthy liver tissue is primarily achieved via the portal vein (vena portae), whereas the liver artery (Arteria hepatica) supplies most primary tumours. After intravenous injection of a bolus of contrast agent, it is accordingly possible to observe a time delay between the signal rise of the healthy liver parenchyma and of the tumour.

In the case of the contrast enhancement achieved by Primovist® during the distribution phase, what is observed are typical perfusion patterns which provide information for the characterization of the lesions. Depicting the wash-in behaviour, the wash-out behaviour and the vascularization helps to characterize the lesion types and to determine the spatial relationship between tumour and blood vessels.

In the case of T1-weighted images, Primovist® leads, 10-20 minutes after the injection (in the hepatobiliary phase), to a distinct signal enhancement in the healthy liver parenchyma, whereas lesions containing no hepatocytes or only a few hepatocytes, for example metastases or moderately to poorly differentiated hepatocellular carcinomas (HCCs), appear as darker regions.

However, the blood vessels also appear as dark regions in the hepatobiliary phase, meaning that, in the MRI images which are generated during the hepatobiliary phase, it is generally not possible to differentiate liver lesions and blood vessels solely on the basis of the contrast. A differentiation between liver lesions and blood vessels can only be achieved in connection with further MRI images, for example of the dynamic phase (in which the blood vessels are highlighted), or else with the aid of MRI images without a contrast enhancement caused by a contrast agent. However, if, for example, an MRI image acquisition method shortened for an examination object is used, for example if a contrast agent is already administered for a certain time span prior to the MRI image acquisition in order to directly acquire MRI images within the hepatobiliary phase and then—after a second administration of contrast agent—MRI images of the dynamic phase are created, it is no longer possible to create an MRI image without contrast enhancement (native MRI image) in the same MRI image acquisition process.

The present disclosure provides, in a first aspect, a method comprising the steps of;
receiving at least one first MRI image of an examination object, the at least one first MRI image showing a liver or a portion of a liver of the examination object, blood vessels in the liver being depicted with contrast enhancement as a result of a contrast agent,
receiving at least one second MRI image of the same examination object, the at least one second MRI image showing the same liver or the same portion of the liver, healthy liver cells being depicted with contrast enhancement as a result of a contrast agent,
feeding the received MRI images to a prediction model, the prediction model having been trained by means of supervised learning to predict, on the basis of MRI images showing a liver or a portion of a liver of an examination object, in which the blood vessels in the liver are depicted with contrast enhancement as a result of a contrast agent, and on the basis of MRI images of the same liver or the same portion of the liver of the same examination object, in which healthy liver cells are depicted with contrast enhancement as a result of a contrast agent, one or more MRI images showing the liver or a portion of the liver of the examination object without a contrast enhancement caused by a contrast agent,
receiving from the prediction model one or more predicted MRI images showing the liver or a portion of the liver of the examination object without a contrast enhancement caused by a contrast agent,
displaying and/or outputting the one or more predicted MRI images and/or storing the one or more predicted MRI images in a data storage medium.

The present disclosure further provides a system comprising

a receiving unit,

a control and calculation unit, and

an output unit,
the control and calculation unit being configured to prompt the receiving unit to receive at least one first MRI image of an examination object, the at least one first MRI image showing a liver or a portion of a liver of the examination object, blood vessels in the liver being depicted with contrast enhancement as a result of a contrast agent,
the control and calculation unit being configured to prompt the receiving unit to receive at least one second MRI image of an examination object, the at least one second MRI image showing the same liver or the same portion of the liver, healthy liver cells being depicted with contrast enhancement as a result of a contrast agent,
the control and calculation unit being configured to predict one or more MRI images on the basis of the received MRI images, the one or more predicted MRI images showing the liver or a portion of the liver of the examination object without a contrast enhancement caused by a contrast agent,
the control and calculation unit being configured to prompt the output unit to display the one or more predicted MRI images, to output them or to store them in a data storage medium.

The present disclosure further provides a computer program product comprising a computer program which can be loaded into a memory of a computer, where it prompts the computer to execute the following steps:
receiving at least one first MRI image of an examination object, the at least one first MRI image showing a liver or a portion of a liver of the examination object, blood vessels in the liver being depicted with contrast enhancement as a result of a contrast agent,
receiving at least one second MRI image of the same examination object, the at least one second MRI image showing the same liver or the same portion of the liver, healthy liver cells being depicted with contrast enhancement as a result of a contrast agent,
feeding the received MRI images to a prediction model, the prediction model having been trained by means of supervised learning to predict, on the basis of MRI images showing a liver or a portion of a liver of an examination object and in which the blood vessels in the liver are depicted with contrast enhancement as a result of a contrast agent, and on the basis of MRI images of the same liver or the same portion of the liver of the same examination object, in which healthy liver cells are depicted with contrast enhancement as a result of a contrast agent, one or more MRI images showing the liver or a portion of the liver of the examination object without a contrast enhancement caused by a contrast agent,
receiving one or more predicted MRI images showing the liver or a portion of the liver of the examination object without a contrast enhancement caused by a contrast agent, as output from the prediction model,
displaying and/or outputting the one or more predicted MRI images and/or storing the one or more predicted MRI images in a data storage medium.

The present disclosure further provides for the use of a contrast agent in an MRI method, the MRI method comprising the following steps:
administering the contrast agent, the contrast agent spreading in a liver of an examination object,
generating at least one first MRI image, the at least one first MRI image showing the liver or a portion of the liver of the examination object, blood vessels in the liver being depicted with contrast enhancement as a result of the contrast agent,
generating at least one second MRI image, the at least one second MRI image showing the same liver or the same portion of the liver, healthy liver cells being depicted with contrast enhancement as a result of the contrast agent,
feeding the generated MRI images to a prediction model, the prediction model having been trained by means of supervised learning to predict, on the basis of MRI images showing a liver or a portion of a liver of an examination object and in which the blood vessels in the liver are depicted with contrast enhancement as a result of a contrast agent, and on the basis of MRI images of the same liver or the same portion of the liver of the same examination object, in which healthy liver cells are depicted with contrast enhancement as a result of a contrast agent, one or more MRI images showing the liver or a portion of the liver of the examination object without a contrast enhancement caused by a contrast agent,
receiving one or more predicted MRI images showing the liver or a portion of the liver of the examination object without a contrast enhancement caused by a contrast agent, as output from the prediction model,
displaying and/or outputting the one or more predicted MRI images and/or storing the one or more predicted MRI images in a data storage medium.

Further provided is a contrast agent for use in an MRI method, the MRI method comprising the following steps:
administering the contrast agent, the contrast agent spreading in a liver of an examination object,
generating at least one first MRI image, the at least one first MRI image showing the liver or a portion of the liver of the examination object, blood vessels in the liver being depicted with contrast enhancement as a result of the contrast agent,
generating at least one second MRI image, the at least one second MRI image showing the same liver or the same portion of the liver, healthy liver cells being depicted with contrast enhancement as a result of the contrast agent,
feeding the generated MRI images to a prediction model, the prediction model having been trained by means of supervised learning to predict, on the basis of MRI images showing a liver or a portion of a liver of an examination object and in which the blood vessels in the liver are depicted with contrast enhancement as a result of a contrast agent, and on the basis of MRI images of the same liver or the same portion of the liver of the same examination object, in which healthy liver cells are depicted with contrast enhancement as a result of a contrast agent, one or more MRI images showing the liver or a portion of the liver of the examination object without a contrast enhancement caused by a contrast agent,
receiving one or more predicted MRI images showing the liver or a portion of the liver of the examination object without a contrast enhancement caused by a contrast agent, as output from the prediction model,
displaying and/or outputting the one or more predicted MRI images and/or storing the one or more predicted MRI images in a data storage medium.

Further provided is a kit comprising a contrast agent and a computer program product according to the disclosure.

The disclosure will be more particularly elucidated below without distinguishing between the subjects of the disclosure (method, system, computer program product, use, contrast agent for use, kit). On the contrary, the following elucidations are intended to apply analogously to all the subjects of the disclosure, irrespective of in which context (method, system, computer program product, use, contrast agent for use, kit) they occur.

If steps are stated in an order in the present description or in the claims, this does not necessarily mean that the disclosure is restricted to the stated order. On the contrary, it is conceivable that the steps are also executed in a different order or else in parallel to one another, unless one step builds upon another step, this absolutely requiring that the building step be executed subsequently (this being, however, clear in the individual case). The stated orders are thus preferred embodiments of the disclosure.

The present disclosure generates one or more artificial MRI images of a liver or of a portion of a liver of an examination object, the one or more MRI images showing the liver or the portion of the liver without a contrast enhancement caused by a contrast agent. The artificial MRI image(s) is/are created on the basis of MRI images which were all recorded with a contrast enhancement caused by a contrast agent. The artificial MRI image(s) can be created with the aid of a self-learning algorithm and imitate(s) MRI image(s) of the liver or of a portion of the liver of the examination object which was not contrast-enhanced by administration of a contrast agent.

The “examination object” is usually a living being, preferably a mammal, very particularly preferably a human.

A portion of the examination object is subjected to a contrast-enhanced magnetic resonance imaging examination. The “examination region”, also called image volume (field of view, FOV), is in particular a volume which is imaged in the magnetic resonance images. The examination region is typically defined by a radiologist, for example on an overview image (localizer). It is self-evident that the examination region can, alternatively or additionally, also be defined automatically, for example on the basis of a selected protocol. The examination region comprises at least one portion of the liver of the examination object.

The examination region is introduced into a basic magnetic field.

A contrast agent which spreads in the examination region is administered to the examination object. The contrast agent is preferably administered intravenously (e.g. into an arm vein) as a bolus using dosing based on body weight.

A “contrast agent” is understood to mean a substance or substance mixture, the presence of which in a magnetic resonance measurement leads to an altered signal. Preferably, the contrast agent leads to a shortening of the T1 relaxation time and/or of the T2 relaxation time.

Preferably, the contrast agent is a hepatobiliary contrast agent such as, for example, Gd-EOB-DTPA or Gd-BOPTA.

In a particularly preferred embodiment, the contrast agent is a substance or a substance mixture with gadoxetic acid or a gadoxetic acid salt as contrast-enhancing active substance. Very particular preference is given to the disodium salt of gadoxetic acid (Gd-EOB-DTPA disodium).

The examination region is subjected to an MRI method and, in the course of this, MRI images are generated (measured) which show the examination region during the examination phase.

The measured MRI images can be present as two-dimensional images showing a sectional plane through the examination object. The measured MRI images can be present as a stack of two-dimensional images, with each individual image of the stack showing a different sectional plane. The measured MRI images can be present as three-dimensional images (3D images). In the interests of simpler illustration, the disclosure will be elucidated at some points in the present description on the basis of the presence of two-dimensional MRI images, without any wish, however, to restrict the disclosure to two-dimensional MRI images. It is clear to a person skilled in the art how it is possible to apply what is respectively described to stacks of two-dimensional images and to 3D images (see, in relation to this, for example M. Reisler, W. Semmler: Magnetresonanztomographie [Magnetic resonance imaging], Springer Verlag, 3rd edition, 2002, ISBN: 978-3-642-63076-7).

After the intravenous administration of a hepatobiliary contrast agent in the form of a bolus, the contrast agent reaches the liver first via the arteries. These are depicted with contrast enhancement in the corresponding MRI images. The phase in which the liver arteries are depicted with contrast enhancement in MRI images is referred to as “arterial phase”. Said phase starts immediately after the administration of the contrast agent and usually lasts 15 to 25 seconds.

Subsequently, the contrast agent reaches the liver via the liver veins. Whereas the contrast in the liver arteries is already decreasing, the contrast in the liver veins is reaching a maximum. The phase in which the liver veins are depicted with contrast enhancement in MRI images is referred to as “venous phase”. Said phase can already start during the arterial phase and overlap therewith. Usually, said phase starts 20 to 30 seconds after the intravenous administration and usually lasts 40 to 60 seconds.

Following the venous phase is the “late phase”, in which the contrast in the liver arteries falls further and the contrast in the liver veins likewise falls and the contrast in the healthy liver cells gradually rises. Said phase usually starts 70 to 90 seconds after the administration of the contrast agent and usually lasts 100 to 120 seconds.

The arterial phase, the venous phase and the late phase are also referred to collectively as “dynamic phase”.

10-20 minutes after its injection, a hepatobiliary contrast agent leads to a distinct signal enhancement in the healthy liver parenchyma. Said phase is referred to as “hepatobiliary phase”. The contrast agent is eliminated only slowly from the liver cells; accordingly, the hepatobiliary phase can last for two hours and longer.

The stated phases are, for example, described in more detail in the following publications: J. Magn. Reson. Imaging, 2012, 35(3): 492-511, doi:10.1002/jmri.22833; Clujul Medical, 2015, Vol. 88 no. 4: 438-448, DOI: 10.15386/cjmed-414; Journal of Hepatology, 2019, Vol. 71: 534-542, (http://dx.doi.org/10.1016/j.jhep.2019.05.005).

In this description, “first MRI image” refers to an MRI image in which blood vessels which are depicted with contrast enhancement as a result of a contrast agent are identifiable. Preferably, the at least one first MRI image is at least one MRI image which was measured during the dynamic phase. Particular preference is given to, in each case, at least one MRI image which was measured during the arterial phase, the venous phase and/or during the late phase. Very particular preference is given to, in each case, at least one MRI image which was measured during the arterial, venous and late phase. Preferably, the at least one first MRI image is a T1-weighted depiction.

When using a paramagnetic contrast agent, the blood vessels are characterized by a high signal intensity in the at least one first MRI image owing to the contrast enhancement (high-signal depiction). Those (continuous) structures within a first MRI image that have a signal intensity within an empirically ascertainable range can thereby be assigned to blood vessels. This means that, with the at least one first MRI image, there is information about where blood vessels are depicted in the MRI images or which structures in the MRI images can be attributed to blood vessels (arteries and/or veins).

In this description, “second MRI image” refers to an MRI image showing the examination region during the hepatobiliary phase. During the hepatobiliary phase, the healthy liver tissue (parenchyma) is depicted with contrast enhancement. Those (continuous) structures within a second MRI image that have a signal intensity within an empirically ascertainable range can thus be assigned to healthy liver cells. Thus, the at least one second MRI image contains information as to where in the MRI images healthy liver cells are depicted or what structures in the MRI images can be attributed to healthy liver cells. Preferably, the at least one second MRI image is a T1-weighted depiction.

The MRI image acquisitions of the dynamic and the hepatobiliary phase of the liver extend over a comparatively long time span. Over said time span, movements by the patient should be avoided in order to minimize movement artefacts in the MRI image. The lengthy restriction of movement can be unpleasant for a patient. Therefore, what is now established are shortened MRI image acquisition procedures, in which a contrast agent is already administered to the examination object for a certain time span (i.e. 10 to 20 minutes) prior to the MRI image acquisition in order to be able to directly acquire MRI images within the hepatobiliary phase. MRI images of the dynamic phase are then acquired in the same MRI image acquisition process after administration of a second dose of the contrast agent.

In comparison with a conventional MRI image acquisition process, the MRI residence time of a patient or an examination object is distinctly shorter as a result. Therefore, the disclosure preferably involves recording the at least one MRI image of the liver or the portion of the liver in the hepatobiliary phase after a (first) administration of a first contrast agent into the examination object and recording at least one further MRI image of the same liver or the portion of the same liver in the dynamic phase after administration of a second contrast agent or a second administration of the first contrast agent into the same examination object. The first contrast agent in this connection is a hepatobiliary, paramagnetic contrast agent. The second contrast agent can also be an extracellular, paramagnetic contrast agent.

The “first MRI image” and the “second MRI image” are fed to a prediction model. The prediction model is a model which is configured to predict, on the basis of the received MRI images, one or more MRI images showing the liver or a portion of the liver of the examination object without a contrast enhancement caused by a contrast agent.

In this connection, the term “prediction” means that the MRI images showing the liver or a portion thereof of an examination object without contrast enhancement caused by a contrast agent are calculated using the MRI images showing the same examination region with contrast enhancement caused by a contrast agent.

The prediction model was preferably created with the aid of a self-learning algorithm in a supervised machine learning process. Learning is achieved by using training data comprising a multiplicity of MRI images of the dynamic phase and of the hepatobiliary phase of the liver or of a portion of the liver of an examination object. Furthermore, use was preferably also made of training data which were created from MRI images of the same liver or portion of a liver of the same examination object and in which there was no contrast enhancement, i.e. which were generated without administration of a contrast agent.

The self-learning algorithm generates, during machine learning, a statistical model which is based on the training data. This means that the examples are not simply learnt by heart, but that the algorithm “recognizes” patterns and regularities in the training data. The algorithm can thus also assess unknown data. Validation data can be used to test the quality of the assessment of unknown data.

The self-learning algorithm is trained by means of supervised learning, i.e. MRI images with contrast enhancement in the dynamic phase and of the hepatobiliary phase are presented successively to the algorithm and it is informed of which non-contrast-enhanced MRI images are associated with these contrast-enhanced MRI images. The algorithm then learns a relationship between the MRI images with contrast enhancement and the MRI images without contrast enhancement in order to predict one or more MRI images without contrast enhancement or MRI images with contrast enhancement.

Self-learning algorithms trained by means of supervised learning are widely described in the prior art (see, for example, C. Perez: Machine Learning Techniques: Supervised Learning and Classification, Amazon Digital Services LLC-Kdp Print Us, 2019, ISBN 1096996545, 9781096996545).

Preferably, the prediction model is an artificial neural network.

Such an artificial neural network comprises at least three layers of processing elements: a first layer with input neurons (nodes), an N-th layer with at least one output neuron (nodes) and N-2 inner layers, where N is a natural number and greater than 2.

The input neurons serve to receive digital MRI images as input values. Normally, there is one input neuron for each pixel or voxel of a digital MRI image. There can be additional input neurons for additional input values (e.g. information about the examination region, about the examination object and/or about conditions which prevailed when generating the MRI images).

In such a network, the output neurons serve to generate a third artificial MRI image for a first and a second MRI image. The processing elements of the layers between the input neurons and the output neurons are connected to one another in a predetermined pattern with predetermined connection weights.

Preferably, the artificial neural network is a so-called convolutional neural network (CNN for short).

A convolutional neural network is capable of processing input data in the form of a matrix. This makes it possible to use digital MRI images depicted as a matrix (e.g. width×height×colour channels) as input data. By contrast, a normal neural network, for example in the form of a multilayer perceptron (MLP), requires a vector as input, i.e. to use an MRI image as input, the pixels or voxels of the MRI image would have to be rolled out successively in a long chain. As a result, normal neural networks are, for example, not capable of recognizing objects in an MRI image independently of the position of the object in the MRI image. The same object at a different position in the MRI image would have a completely different input vector.

A CNN consists essentially of filters (convolutional layer) and aggregation layers (pooling layer) which are repeated alternately and, at the end, of one layer or multiple layers of “normal” completely connected neurons (dense/fully connected layer).

When analysing sequences (sequences of MRI image), space and time can be treated as equivalent dimensions and, for example, processed via 3D folds. This has been shown in the papers by Baccouche et al. (Sequential Deep Learning for Human Action Recognition; International Workshop on Human Behavior Understanding, Springer 2011, pages 29-39) and Ji et al. (3D Convolutional Neural Networks for Human Action Recognition, IEEE Transactions on Pattern Analysis and Machine Intelligence, 35(1), 221-231). Furthermore, it is possible to train different networks which are responsible for time and space and to lastly merge the features, as described in publications by Karpathy et al. (Large-scale Video Classification with Convolutional Neural Networks; Proceedings of the IEEE conference on Computer Vision and Pattern Recognition, 2014, pages 1725-1732) and Simonyan & Zisserman (Two-stream Convolutional Networks for Action Recognition in Videos; Advances in Neural Information Processing Systems, 2014, pages 568-576).

Recurrent Neural Networks (RNNs) are a family of so-called feedforward neural networks which contain feedback connections between layers. RNNs allow the modelling of sequential data by common utilization of parameter data via different parts of the neural network. The architecture for an RNN contains cycles. The cycles represent the influence of a current value of a variable on its own value at a future time point, since at least a portion of the output data from the RNN is used as feedback for processing subsequent inputs in a sequence.

Details can be gathered from the prior art (see, for example: S. Khan et al.: A Guide to Convolutional Neural Networks for Computer Vision, Morgan & Claypool Publishers 2018, ISBN 1681730227, 9781681730226).

The training of the neural network can, for example, be carried out by means of a backpropagation method. In this connection, what is striven for, for the network, is a mapping of given input vectors onto given output vectors that is as reliable as possible. The mapping quality is described by an error function. The goal is to minimize the error function. In the case of the backpropagation method, an artificial neural network is taught by altering the connection weights.

In the trained state, the connection weights between the processing elements contain information regarding the relationship between the contrast-enhanced MRI images of the dynamic and hepatobiliary phase and MRI images without contrast enhancement that can be used in order to predict one or more MRI images which show an examination region without contrast enhancement and which are calculated only by means of contrast-enhanced MRI images of the examination region.

A cross-validation method can be used in order to divide the data into training and validation data sets. The training data set is used in the backpropagation training of network weights. The validation data set is used in order to check the accuracy of prediction with which the trained network can be applied to unknown pluralities of MRI images.

As already indicated, further information about the examination object, about the examination region and/or about examination conditions can also be used for training, validation and prediction.

Examples of information about the examination object are: sex, age, weight, height, anamnesis, nature and duration and amount of medicaments already ingested, blood pressure, central venous pressure, breathing rate, serum, albumin, total bilirubin, blood sugar, iron content, breathing capacity and the like. These can, for example, also be gathered from a database or an electronic patient file.

Examples of information about the examination region are: pre-existing conditions, operations, partial resection, liver transplantation, iron liver, fatty liver and the like.

It is conceivable that the received MRI images are subjected to a retrospective movement correction before they are fed to the prediction model. Such a movement correction ensures that a pixel or voxel of a first MRI image shows the same examination region as the corresponding pixel or voxel of a second, temporally downstream MRI image. Movement correction methods are described in the prior art (see, for example: EP3118644, EP3322997, US20080317315, US20170269182, US20140062481, EP2626718).

The present disclosure provides a system which makes it possible to execute the method according to the disclosure.

It is conceivable that the stated units are components of a single computer system; however, it is also conceivable that the stated units are components of multiple separate computer systems which are connected to one another via a network in order to transmit data and/or control signals from one unit to another unit.

A “computer system” is a system for electronic data processing that processes data by means of programmable calculation rules. Such a system usually comprises a “computer”, that unit which comprises a processor for carrying out logical operations, and also peripherals.

In computer technology, “peripherals” refer to all devices which are connected to the computer and serve for the control of the computer and/or as input and output devices. Examples thereof are monitor (screen), printer, scanner, mouse, keyboard, drive, camera, microphone, loudspeaker, etc. Internal ports and expansion cards are, too, considered to be peripherals in computer technology.

Computer systems of today are frequently divided into desktop PCs, portable PCs, laptops, notebooks, netbooks and tablet PCs and so-called handhelds (e.g. smartphone); all these systems can be utilized for carrying out the disclosure.

Inputs into the computer system are achieved via input means such as, for example, a keyboard, a mouse, a microphone, a touch-sensitive display and/or the like.

The system according to the disclosure is configured to receive at least one first MRI image with contrast enhancement of the hepatobiliary phase and at least one second MRI image with contrast enhancement of the dynamic phase and to generate (to predict, to calculate), on the basis of these data and optionally further data, one or more MRI images showing the examination region, i.e. the liver or parts thereof, without contrast enhancement.

The control and calculation unit serves for the control of the receiving unit, the coordination of the data and signal flows between various units, and the processing and generation of MRI images. It is conceivable that multiple control and calculation units are present.

The receiving unit serves for the receiving of MRI images. The MRI images can, for example, be transmitted from a magnetic resonance system or be read from a data storage medium. The magnetic resonance system can be a component of the system according to the disclosure. However, it is also conceivable that the system according to the disclosure is a component of a magnetic resonance system.

The at least one first MRI image and the at least one second MRI image and optionally further data are transmitted from the receiving unit to the control and calculation unit.

The control and calculation unit is configured to predict, on the basis of the MRI images showing an examination region with contrast enhancement of the dynamic and the hepatobiliary phase, one or more MRI images, the predicted MRI images showing the examination region without contrast enhancement. Preferably, what can be loaded into a memory of the control and calculation unit is a prediction model which is used to calculate the MRI images without contrast enhancement. The prediction model was preferably generated (trained) with the aid of a self-learning algorithm by means of supervised learning.

Via the output unit, the at least one predicted MRI image can be displayed (e.g. on a monitor), be outputted (e.g. via a printer) or be stored in a data storage medium.

A further embodiment of the disclosure concerns the use of a contrast agent or a contrast agent for use in an MRI method, the MRI method comprising the following steps:
administering the contrast agent, the contrast agent spreading in a liver of an examination object,
generating at least one first MRI image, the at least one first MRI image showing the liver or a portion of the liver of the examination object, blood vessels in the liver being depicted with contrast enhancement as a result of the contrast agent,
generating at least one second MRI image, the at least one second MRI image showing the same liver or the same portion of the liver, healthy liver cells being depicted with contrast enhancement as a result of the contrast agent,
feeding the generated MRI images to a prediction model, the prediction model having been trained by means of supervised learning to predict, on the basis of MRI images showing a liver or a portion of a liver of an examination object and in which the blood vessels in the liver are depicted with contrast enhancement as a result of a contrast agent, and on the basis of MRI images of the same liver or the same portion of the liver of the same examination object, in which healthy liver cells are depicted with contrast enhancement as a result of a contrast agent, one or more MRI images showing the liver or a portion of the liver of the examination object without a contrast enhancement caused by a contrast agent,
receiving one or more predicted MRI images showing the liver or a portion of the liver of the examination object without a contrast enhancement caused by a contrast agent, as output from the prediction model,
displaying and/or outputting the one or more predicted MRI images and/or storing the one or more predicted MRI images in a data storage medium.

In a preferred variant, the at least one “second MRI image” is generated after a (first) administration of a first contrast agent into the examination object and the at least one “first MRI image” is generated after a second administration of the first contrast agent or an administration of a second contrast agent into the same examination object. This means that the above-defined “second MRI image” is, in terms of time, generated before the above-defined “first MRI image”.

The disclosure is more particularly elucidated below with reference to figures, without wishing to restrict the disclosure to the features or combinations of features that are shown in the figures, where:

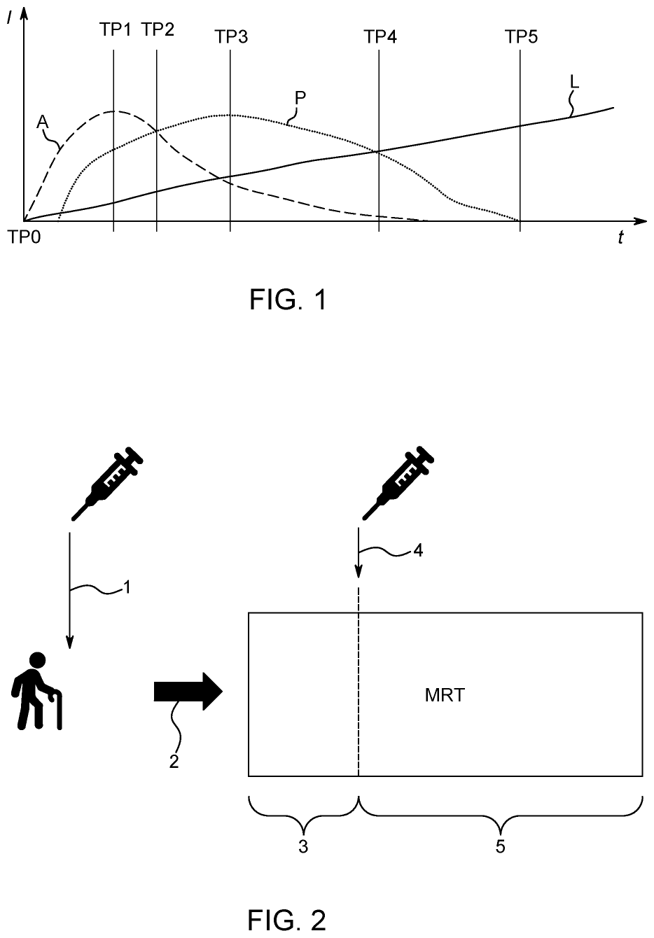

FIG. 1 shows schematically the temporal profile of the concentrations of contrast agent in the liver arteries (A), the liver veins (P) and the liver cells (L). The concentrations are depicted in the form of the signal intensities I in the stated areas (liver arteries, liver veins, liver cells) in the magnetic resonance measurement as a function of the time t. Upon an intravenous bolus injection, the concentration of the contrast agent rises in the liver arteries (A) first of all (dashed curve). The concentration passes through a maximum and then drops. The concentration in the liver veins (P) rises more slowly than in the liver arteries and reaches its maximum later (dotted curve). The concentration of the contrast agent in the liver cells (L) rises slowly (continuous curve) and reaches its maximum only at a very much later time point (not depicted in FIG. 1). A few characteristic time points can be defined: At time point TP0, contrast agent is administered intravenously as a bolus. At time point TP1, the concentration (the signal intensity) of the contrast agent in the liver arteries reaches its maximum. At time point TP2, the curves of the signal intensities for the liver arteries and the liver veins intersect. At time point TP3, the concentration (the signal intensity) of the contrast agent in the liver veins passes through its maximum. At time point TP4, the curves of the signal intensities for the liver veins and the liver cells intersect. At time point TP5, the concentrations in the liver arteries and the liver veins have dropped to a level at which they no longer cause a measurable contrast enhancement.

FIG. 2 shows schematically an example of a shortened MRI image acquisition procedure. In a shortened MRI image acquisition procedure, a contrast agent is first administered (1). The examination object is introduced to the MRI after a certain waiting period, for example 10 to 20 minutes (2). Thereafter, the MRI process is started and an MRI of the liver or a portion thereof in the hepatobiliary phase is first carried out (3). Thereafter, a further intravenous bolus injection (4) is administered to the examination object and an MRI of the liver or a portion thereof in the dynamic phase is directly subsequently carried out.

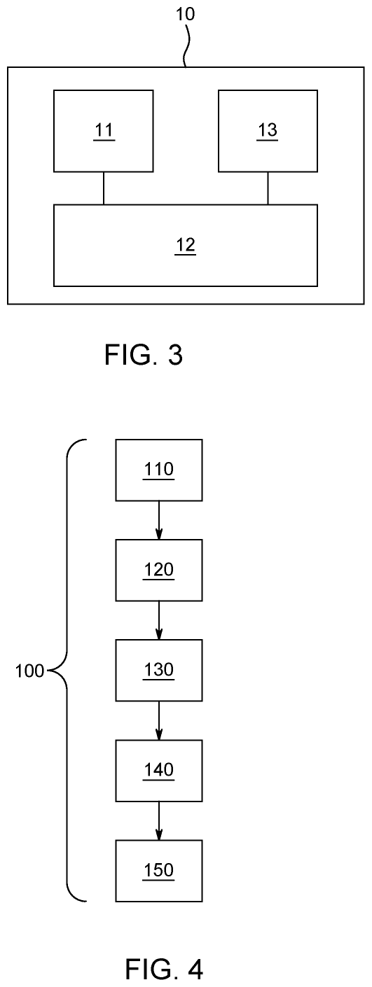

FIG. 3 shows schematically a preferred embodiment of the system according to the disclosure. The system (10) comprises a receiving unit (11), a control and calculation unit (12) and an output unit (13).

The control and calculation unit (12) is configured to prompt the receiving unit (11) to receive at least one first MRI image of an examination object, the at least one first MRI image showing a liver or a portion of a liver of the examination object, blood vessels in the liver being depicted with contrast enhancement as a result of a contrast agent.

The control and calculation unit (12) is further configured to prompt the receiving unit (11) to receive at least one second MRI image of an examination object, the at least one second MRI image showing the same liver or the same portion of the liver, healthy liver cells being depicted with contrast enhancement as a result of a contrast agent.

The control and calculation unit (12) is further configured to predict one or more MRI images on the basis of the received MRI images, the one or more predicted MRI images showing the liver or a portion of the liver of the examination object without a contrast enhancement caused by a contrast agent.

The control and calculation unit (12) is further configured to prompt the output unit (13) to display the at least one predicted MRI image, to output it or to store it in a data storage medium.

FIG. 4 shows schematically and exemplarily one embodiment of the method according to the disclosure. The method (100) comprises the steps:
(110) receiving at least one first MRI image of an examination object, the at least one first MRI image showing a liver or a portion of a liver of the examination object, blood vessels in the liver being depicted with contrast enhancement as a result of a contrast agent,
(120) receiving at least one second MRI image of the same examination object, the at least one second MRI image showing the same liver or the same portion of the liver, healthy liver cells being depicted with contrast enhancement as a result of a contrast agent,
(130) feeding the received MRI images to a prediction model, the prediction model having been trained by means of supervised learning to predict, on the basis of MRI images showing a liver or a portion of a liver of an examination object and in which the blood vessels in the liver are depicted with contrast enhancement as a result of a contrast agent, and on the basis of MRI images of the same liver or the same portion of the liver of the same examination object, in which healthy liver cells are depicted with contrast enhancement as a result of a contrast agent, one or more MRI images showing the liver or a portion of the liver of the examination object without a contrast enhancement caused by a contrast agent,
(140) receiving from the prediction model one or more predicted MRI images showing the liver or a portion of the liver of the examination object without a contrast enhancement caused by a contrast agent,
(150) displaying and/or outputting the one or more predicted MRI images and/or storing the one or more predicted MRI images in a data storage medium.

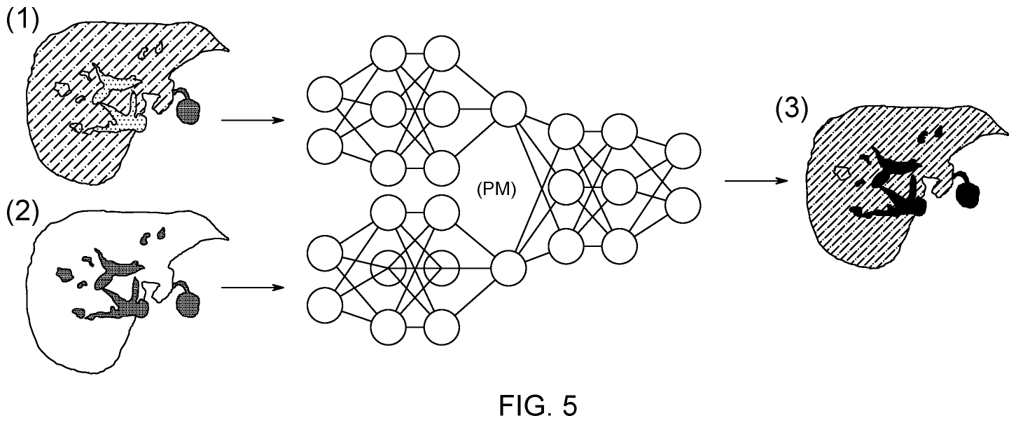

FIG. 5 shows exemplarily and schematically a further embodiment of the present disclosure. A first MRI image (1) is provided, the first MRI image showing a liver or a portion of a liver of an examination object, blood vessels in the liver being depicted with contrast enhancement (signal enhancement) as a result of a contrast agent.

A second MRI image (2) is provided, the second MRI image showing the same liver or the same portion of the liver as the first MRI image, the healthy liver tissue (parenchyma) being depicted with contrast enhancement (signal enhancement) as a result of a contrast agent.

The first MRI image (1) and the second MRI image (2) are fed to a prediction model (PM).

The prediction model (PM) is configured to generate, on the basis of the first MRI image (1) and the second MRI image (2), a third MRI image (3) showing an MRI image without a contrast enhancement caused by a contrast agent.

The prediction model was preferably created with the aid of a self-learning algorithm in a supervised machine learning process with a training data set. The training data set comprises a multiplicity of first MRI images, second MRI images and the associated third MRI images, the third MRI images having actually been recorded, e.g. before administration of a first intravenous bolus of the contrast agent.

The self-learning algorithm generates, during machine learning, a statistical model which is based on the training data. This means that the examples are not simply learnt by heart, but that the algorithm “recognizes” patterns and regularities in the training data. The algorithm can thus also assess unknown data. Validation data can be used to test the quality of the assessment of unknown data.

The self-learning algorithm is trained by means of supervised learning, i.e. first and second MRI images are presented to the algorithm and it is informed of which third MRI images are associated with the particular first and second MRI images. The algorithm then learns a relationship between the MRI images in order to predict (to calculate) third MRI images for unknown first and second MRI images.

Self-learning algorithms trained by means of supervised learning are widely described in the prior art (see, for example, C. Perez: Machine Learning Techniques: Supervised Learning and Classification, Amazon Digital Services LLC-Kdp Print Us, 2019, ISBN 1096996545, 9781096996545).

Preferably, the prediction model is an artificial neural network.

Such an artificial neural network comprises at least three layers of processing elements: a first layer with input neurons (nodes), an N-th layer with at least one output neuron (nodes) and N-2 inner layers, where N is a natural number and greater than 2.

The input neurons serve to receive digital MRI images as input values. Normally, there is one input neuron for each pixel or voxel of a digital MRI image. There can be additional input neurons for additional input values (e.g. information about the examination region, about the examination object and/or about conditions which prevailed when generating the MRI images).

In such a network, the output neurons serve to generate a third MRI image for a first and a second MRI image.

The processing elements of the layers between the input neurons and the output neurons are connected to one another in a predetermined pattern with predetermined connection weights.

Preferably, the artificial neural network is a so-called convolutional neural network (CNN for short).

A convolutional neural network is capable of processing input data in the form of a matrix. This makes it possible to use digital MRI images depicted as a matrix (e.g. width×height×colour channels) as input data. By contrast, a normal neural network, for example in the form of a multilayer perceptron (MLP), requires a vector as input, i.e. to use an MRI image as input, the pixels or voxels of the MRI image would have to be rolled out successively in a long chain. As a result, normal neural networks are, for example, not capable of recognizing objects in an MRI image independently of the position of the object in the MRI image. The same object at a different position in the MRI image would have a completely different input vector.

A CNN consists essentially of filters (convolutional layer) and aggregation layers (pooling layer) which are repeated alternately and, at the end, of one layer or multiple layers of “normal” completely connected neurons (dense/fully connected layer).

Details can be gathered from the prior art (see, for example: S. Khan et al.: A Guide to Convolutional Neural Networks for Computer Vision, Morgan & Claypool Publishers 2018, ISBN 1681730227, 9781681730226).

The training of the neural network can, for example, be carried out by means of a backpropagation method. In this connection, what is striven for, for the network, is a mapping of given input vectors onto given output vectors that is as reliable as possible. The mapping quality is described by an error function. The goal is to minimize the error function. In the case of the backpropagation method, an artificial neural network is taught by altering the connection weights.

In the trained state, the connection weights between the processing elements contain information regarding the relationship between the contrast-enhanced MRI images of the dynamic and hepatobiliary phase and MRI images without contrast enhancement that can be used in order to predict one or more MRI images which show an examination region without contrast enhancement and which are calculated only by means of contrast-enhanced MRI images of the same examination region.

A cross-validation method can be used in order to divide the data into training and validation data sets. The training data set is used in the backpropagation training of network weights. The validation data set is used in order to check the accuracy of prediction with which the trained network can be applied to unknown pluralities of MRI images.

1. A method comprising:
receiving at least one first MRI image of an examination object, the at least one first MRI image showing a liver or a portion of a liver of the examination object, blood vessels in the liver being depicted with contrast enhancement as a result of a contrast agent,
receiving at least one second MRI image of the same examination object, the at least one second MRI image showing the same liver or the same portion of the liver, healthy liver cells being depicted with contrast enhancement as a result of the contrast agent,
feeding the received MRI images to a prediction model, the prediction model having been trained by means of supervised learning to predict, based on MRI images showing a liver or a portion of a liver of an examination object and in which blood vessels in the liver are depicted with contrast enhancement as a result of a contrast agent, and based on MRI images of the same liver or the same portion of the liver of the same examination object, in which healthy liver cells are depicted with contrast enhancement as a result of the contrast agent, one or more MRI images showing the liver or a portion of the liver of the examination object without a contrast enhancement caused by the contrast agent,
receiving from the prediction model one or more predicted MRI images showing the liver or a portion of the liver of the examination object without a contrast enhancement caused by the contrast agent, and
displaying and/or outputting the one or more predicted MRI images and/or storing the one or more predicted MRI images in a data storage medium.

2. The method according to claim 1, wherein the at least one first MRI image is a T1-weighted depiction of the liver or the portion of the liver in a dynamic phase after administration of a hepatobiliary, paramagnetic contrast agent.

3. The method according to claim 2, wherein the at least one first MRI image is an MRI image which:
(i) shows the liver or a portion of the liver of the examination object during an arterial phase,
(ii) shows the same liver or the same portion of the liver of the same examination object during a venous phase, and
(iii) shows the same liver or the same portion of the liver of the same examination object during a late phase.

4. The method according to claim 1, wherein the at least one second MRI image is a T1-weighted depiction of the liver or the portion of the liver in a hepatobiliary phase after administration of a hepatobiliary, paramagnetic contrast agent or of an extracellular, paramagnetic contrast agent.

5. The method according to claim 2, wherein the at least one second MRI image having a T1-weighted depiction of the liver or the portion of the liver in a hepatobiliary phase after a first administration of the hepatobiliary, paramagnetic contrast agent into the examination object is recorded, and the at least one first MRI image having a T1-weighted depiction of the same liver or the portion of the same liver in the dynamic phase after a second administration of the hepatobiliary, paramagnetic contrast agent or of an extracellular, paramagnetic contrast agent into the same examination object is recorded.

6. The method according to claim 1, wherein the contrast agent is a substance or a substance mixture with gadoxetic acid or a gadoxetic acid salt as contrast-enhancing active substance, preferably the disodium salt of gadoxetic acid.

7. The method according to claim 1, wherein the examination object is a mammal, preferably a human.

8. The method according to claim 1, wherein the prediction model is an artificial neural network.

9. A system comprising:
a receiving unit,
a control and calculation unit, and
an output unit, wherein
the control and calculation unit being configured to prompt the receiving unit to receive at least one first MRI image of an examination object, the at least one first MRI image showing a liver or a portion of a liver of the examination object, blood vessels in the liver being depicted with contrast enhancement as a result of a contrast agent,
the control and calculation unit being configured to prompt the receiving unit to receive at least one second MRI image of the examination object, the at least one second MRI image showing the same liver or the same portion of the liver, healthy liver cells being depicted with contrast enhancement as a result of the contrast agent,
the control and calculation unit being configured to predict one or more MRI images based on the received MRI images, the one or more predicted MRI images showing the liver or a portion of the liver of the examination object without a contrast enhancement caused by the contrast agent, and
the control and calculation unit being configured to prompt the output unit to display the one or more predicted MRI images, to output them or to store them in a data storage medium.

10. A computer program product comprising a computer program which can be loaded into a memory of a computer, where it prompts the computer to execute the following:
receiving at least one first MRI image of an examination object, the at least one first MRI image showing a liver or a portion of a liver of the examination object, blood vessels in the liver being depicted with contrast enhancement as a result of a contrast agent,
receiving at least one second MRI image of the same examination object, the at least one second MRI image showing the same liver or the same portion of the liver, healthy liver cells being depicted with contrast enhancement as a result of the contrast agent,
feeding the received MRI images to a prediction model, the prediction model having been trained by means of supervised learning to predict, based on MRI images showing a liver or a portion of a liver of an examination object and in which blood vessels in the liver are depicted with contrast enhancement as a result of a contrast agent, and based on MRI images of the same liver or the same portion of the liver of the same examination object, in which healthy liver cells are depicted with contrast enhancement as a result of the contrast agent, one or more MRI images showing the liver or a portion of the liver of the examination object without a contrast enhancement caused by the contrast agent,
receiving one or more predicted MRI images showing the liver or a portion of the liver of the examination object without a contrast enhancement caused by the contrast agent, as output from the prediction model, and
displaying and/or outputting the one or more predicted MRI images and/or storing the one or more predicted MRI images in a data storage medium.

11. The computer program product according to claim 10, wherein, the at least one first MRI image is a T1-weighted depiction of the liver or the portion of the liver in a dynamic phase after administration of a hepatobiliary, paramagnetic contrast agent.

12. Use of a contrast agent in an MRI method, the MRI method comprising:
administering the contrast agent, the contrast agent spreading in a liver of an examination object,
generating at least one first MRI image, the at least one first MRI image showing the liver or a portion of the liver of the examination object, blood vessels in the liver being depicted with contrast enhancement as a result of the contrast agent,
generating at least one second MRI image, the at least one second MRI image showing the same liver or the same portion of the liver, healthy liver cells being depicted with contrast enhancement as a result of the contrast agent,
feeding the generated MRI images to a prediction model, the prediction model having been trained by means of supervised learning to predict, on MRI images showing a liver or a portion of a liver of an examination object and in which the blood vessels in the liver are depicted with contrast enhancement as a result of a contrast agent, and based on MRI images of the same liver or the same portion of the liver of the same examination object, in which healthy liver cells are depicted with contrast enhancement as a result of the contrast agent, one or more MRI images showing the liver or a portion of the liver of the examination object without a contrast enhancement caused by the contrast agent,
receiving one or more predicted MRI images showing the liver or a portion of the liver of the examination object without a contrast enhancement caused by the contrast agent, as output from the prediction model, and
displaying and/or outputting the one or more predicted MRI images and/or storing the one or more predicted MRI images in a data storage medium.

13. (canceled)

14. The contrast agent for use according to claim 12, wherein the contrast agent is a substance or a substance mixture with gadoxetic acid or a gadoxetic acid salt as contrast-enhancing active substance, preferably the disodium salt of gadoxetic acid.

15. A kit comprising a contrast agent according to claim 12, and a computer program product according to claim 10.

16. The computer program product according to claim 11, wherein the at least one first MRI image is an MRI image which
(i) shows the liver or a portion of the liver of the examination object during an arterial phase,
(ii) shows the same liver or the same portion of the liver of the same examination object during a venous phase, and
(iii) shows the same liver or the same portion of the liver of the same examination object during a late phase.

17. The computer program product according to claim 10, wherein the at least one second MRI image is a T1-weighted depiction of the liver or the portion of the liver in a hepatobiliary phase after administration of a hepatobiliary, paramagnetic contrast agent or of an extracellular, paramagnetic contrast agent.

18. The computer program product according to claim 11, wherein the at least one second MRI image having a T1-weighted depiction of the liver or the portion of the liver in a hepatobiliary phase after a first administration of the hepatobiliary, paramagnetic contrast agent into the examination object is recorded, and the at least one first MRI image having a T1-weighted depiction of the same liver or the portion of the same liver in the dynamic phase after a second administration of the hepatobiliary, paramagnetic contrast agent or of an extracellular, paramagnetic contrast agent into the same examination object is recorded.

19. The computer program product according to claim 10, wherein the contrast agent is a substance or a substance mixture with gadoxetic acid or a gadoxetic acid salt as contrast-enhancing active substance, preferably the disodium salt of gadoxetic acid.

20. The computer program product according to claim 10, wherein the prediction model is an artificial neural network.
